(19) United States
(12) Patent Application Publication
(10) Pub. No.: US 2022/0370663 A1
(43) Pub. Date: Nov. 24, 2022

(54) MOBILE TELEPHONE CASE STERILIZING MEANS

(71) Applicant: Zeinab Bazzi, Deerborn, MI (US)

(72) Inventor: Zeinab Bazzi, Deerborn, MI (US)

(21) Appl. No.: 17/324,788

(22) Filed: May 19, 2021

(51) Int. Cl. A61L 2/22; A61L 2/10; A45C 15/00

(52) U.S. Cl. CPC A61L 2/22; A61L 2/10; A45C 15/00; A61L 2202/11; A61L 2202/14; A61L 2202/15; A61L 2202/16; A45C 2011/002

(57) ABSTRACT

A mobile telephone case has sanitizing systems incorporated within a case body capable of projecting sanitizing elements onto front side and back sides of the case for clearing the case. A control means is in electrical communication between the sanitizing systems and a power supply. Sets of nozzles located on front and rear sides pumps the sanitizing elements from a sanitizing solution reservoir in the case. Sets of UV-C LED lamps may also be located on front and rear sides of the case for further sanitizing the phone.

### RELATED APPLICATIONS

This non-provisional application claims priority from provisional application No. 63/073,979.

### I. FIELD OF THE INVENTION

The present invention relates to a selective sterilizing means for a mobile telephone case.

### II. MOTIVATION FOR THE INVENTION

In recent society, there has been a trend toward increased sanitization of everyday objects and surfaces one tends to come in contact with. This trend is evidenced by a quick trip to one's local store where shelves are lined with antibacterial soap, hand sanitizer, anti-bacterial surface cleaner, HEPA air filtration systems and the like. At the same time, pandemics such as COVID-19, are becoming more and more common further reinforcing the importance of reduced contact with common surfaces and items. There is perhaps no greater common item than the typical mobile telephone which is not only handled continuously throughout the day by the user, but touched by others as well, and set upon potentially contaminated surfaces also. Accordingly, there exists a need for a means by which a mobile telephones can be cleaned and decontaminated against germs, bacteria, viruses, and the like, in an easy and effortless manner. The use of the mobile telephone case sterilizing means allows for the sanitizing of mobile telephones and cases in a manner which is not only quick, easy, and effective, but safe as well.

### III. DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a front view of the mobile telephone case 10, according to the preferred embodiment of the present invention; and,

FIG. 2 is a rear view of the mobile telephone case 10, according to the preferred embodiment of the present invention; and,

FIG. 3 is a perspective view of the mobile telephone case 10, according to an alternate embodiment of the present invention; and,

FIG. 4 is an electrical block diagram of the mobile telephone case 10, according to the preferred embodiment of the present invention.

### DESCRIPTIVE KEY

10 mobile telephone case
15 mobile telephone
20 protective border
25 button cover
30 front-mounted nozzle
35 sanitizing solution
40 front-mounted UV-C LED
45 enclosure
50 opening
55 rear-mounted nozzle
60 rear-mounted UV-C LED
65 first pushbutton switch
70 second pushbutton switch
75 reservoir
80 fill port
85 recharging port
90 phone charging port
95 charger regulating circuit
100 rechargeable battery
105 pump motor
110 LED driver/timer circuit

### IV. DESCRIPTION OF THE INVENTION

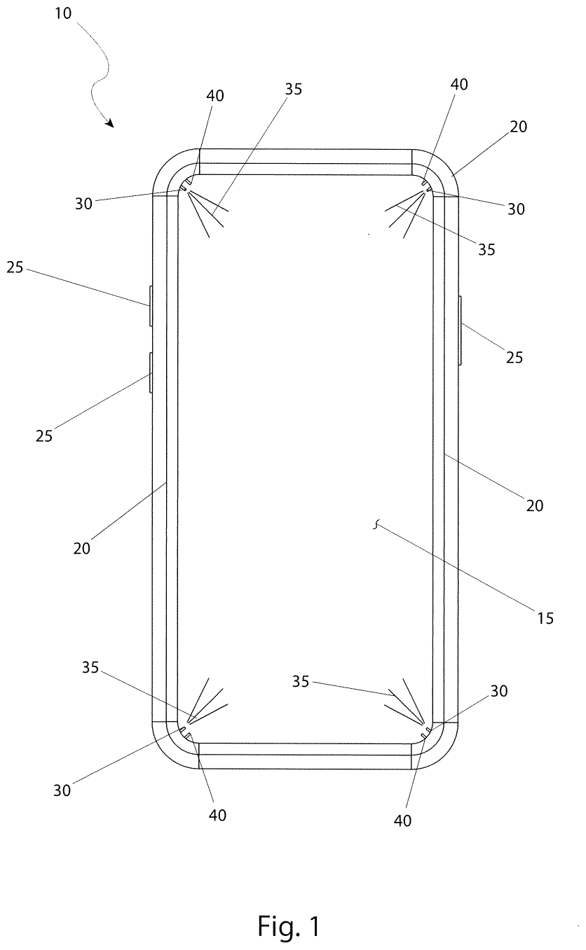

The best mode for carrying out the invention is presented in terms of its preferred embodiment, herein depicted within FIGS. 1 through 4 However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one (1) particular configuration shall be shown and described for purposes of clarity and disclosure and not by way of limitation of scope. All of the implementations described below are exemplary implementations provided to enable persons skilled in the art to make or use the embodiments of the disclosure and are not intended to limit the scope of the disclosure, which is defined by the claims.

The terms “a” and “an” herein do not denote a limitation of quantity, but rather denote the presence of at least one (1) of the referenced items.

### 1. DETAILED DESCRIPTION OF THE FIGURES

Referring now to FIG. 1, a front view of the mobile telephone case 10, according to the preferred embodiment of the present invention is disclosed. The mobile telephone case 10 (herein also described as the “device”) 10, includes of a snap-on protective case for a mobile telephone 15 that provides for periodic sanitation of the device 10 as well as the mobile telephone 15. The device 10 comprises a protective border 20 located around the perimeter of the mobile telephone 15, which provides for the physical protection of the mobile telephone 15 should it be dropped, become in contact with another object, or set upon by another object. The protective border 20 provides for button covers 25 which permits all functionality of the mobile telephone 15 to continue in a non-interfering manner.

The sanitizing process is accomplished by one (1) of two (2) methods. A first method entails a set of front-mounted nozzles 30 which dispense sanitizing solution 35 onto the mobile telephone 15, which are located in each corner of the device 10. Additional components and further description will be provided herein below. A second method utilizes a set of front-mounted UV-C LED's 40 are located in each corner of the device 10. The front-mounted UV-C LED's 40 project ultraviolet light with wavelengths between two hundred to two hundred eighty nanometers (200-280 nm) onto the mobile telephone 15. Light rays in the UV-C wavelength are used to disinfect the mobile telephone 15. The user can utilize either sanitizing method offered by the sanitizing solution 35, which will be described in greater detail herein below.

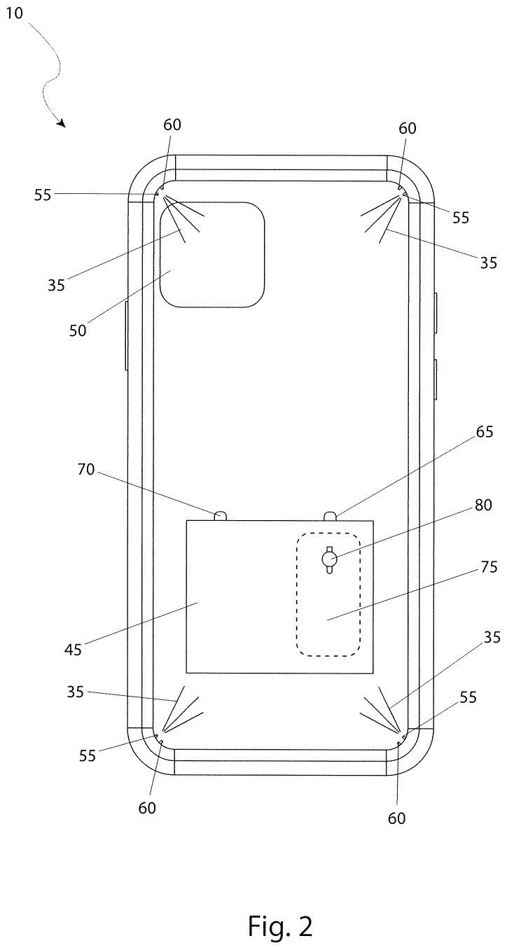

Referring next to FIG. 2, a rear view of the device 10, according to the preferred embodiment of the present invention is depicted. The rear of the mobile telephone 15 is provided with an enclosure 45 which houses various components of the device 10. The overall size of the enclosure 45 is envisioned that it will not interfere with the usage or functionality of the mobile telephone 15. At least one (1) opening is envisioned on the rear of the device 10 to allow for functionality of cameras, LED lights, microphones, speakers, or the like. In a manner similar to the front of the mobile telephone 15, a series of rear-mounted nozzles 55 which dispense sanitizing solution 35 onto the mobile telephone 15 are located in each corner of the device 10. Additional components and further description will be provided herein below. Second, a set of rear-mounted UVC LED's 60 are located in each corner of the device 10. These rear-mounted UV-C LED's 60 project ultraviolet light onto the mobile telephone 15 for disinfecting purposes. The enclosure 45 is provided with a first pushbutton switch 65, which when activated, provide for the dispersal of the sanitizing solution 35 from the front-mounted nozzles 30 (as shown in FIG. 1) and the rear mounted nozzles 55. The enclosure 45 is also provided with a second pushbutton switch 70, which when activated, provide for energization of the front-mounted UV-C LED's 40 and the rear-mounted UV-C LED's 60 for a predetermined time period. The interior of the enclosure 45 is provided with a reservoir 75 (depicted here by dashed lines due to its hidden nature), which holds the sanitizing solution 35. When depleted, the reservoir 75 is manually refilled via a fill port 80 which is opened to allow sanitizing solution 35 to be poured into the reservoir 75. The refilling process interval would vary in frequency depending on the usage of the first pushbutton switch 65.

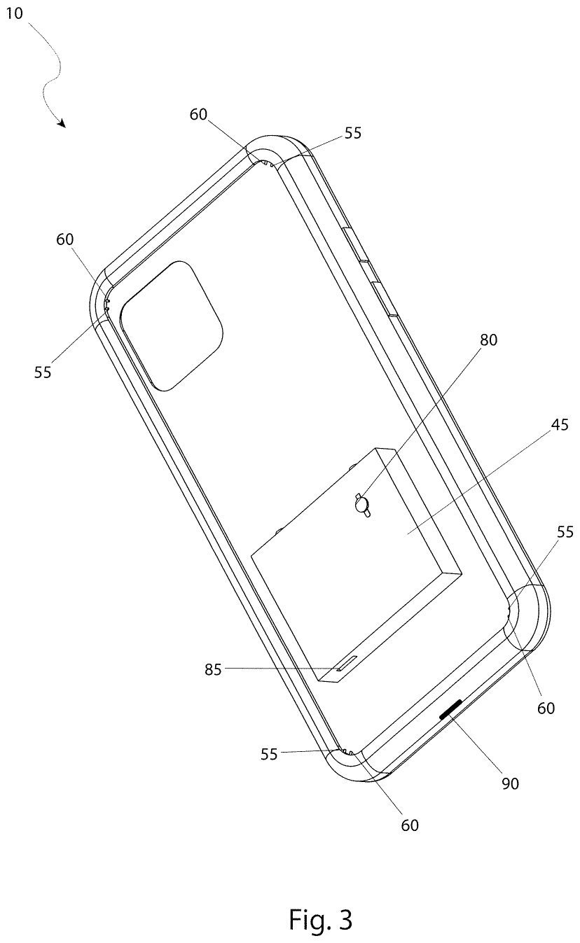

Referring now to FIG. 3, a perspective view of the device 10, according to an alternate embodiment of the present invention is shown. The enclosure 45 with the fill port 80 remain visible along with all of the rear-mounted nozzles 55 and rear-mounted UV-C LED's 60. A recharging port 85 is provided on the bottom of the enclosure 45 to provide electrical charging power for the device 10. Additional detail on the electrical operation of the device 10 will be provided herein below.

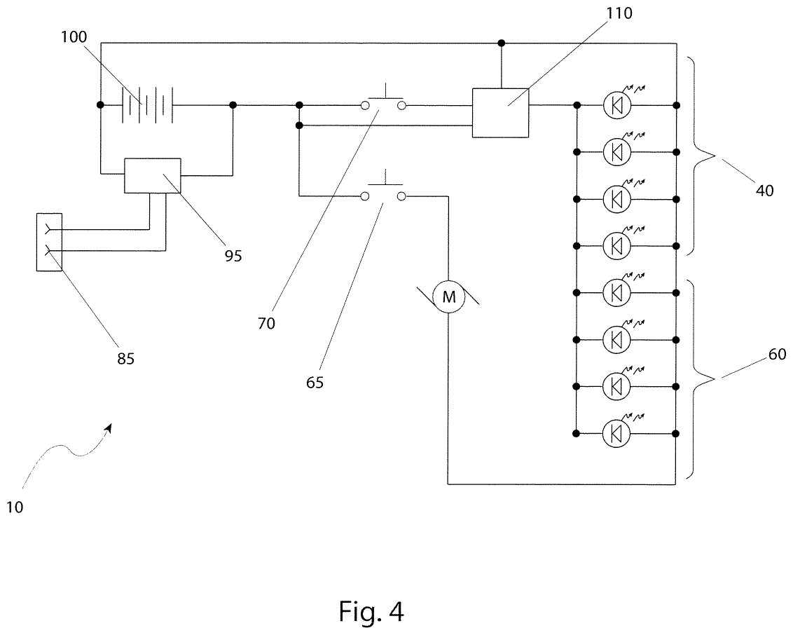

Referring to FIG. 4, an electrical block diagram of the device 10, according to the preferred embodiment of the present invention is disclosed. Electrical power is provided into the device 10 via the recharging port 85 which then passes onto a rechargeable battery 100. Resultant electrical power is then controlled by the first pushbutton switch 65 which passes power onto a pump motor 105. When the pump motor 105 is energized, it pumps sanitizing solution 35 (as shown in FIGS. 1 and 2) out through the front-mounted nozzles 30 and the rear-mounted nozzles 55 (as shown in FIGS. 1 and 2) from the reservoir 75 (as shown in FIG. 2). Resultant power is also controlled by the second pushbutton switch 70 which passes power onto a LED driver/timer circuit 110. The LED driver/timer circuit 110 conditions the electrical power to a suitable voltage and current level to drive the front-mounted UV-C LED's 40 and the rear-mounted UV-C LED's 60 in a parallel circuit arrangement. Additionally, the LED driver/timer circuit 110 keeps the front-mounted UV-C LED's 40 and the rear-mounted UV-C LED's 60 energized for a suitable period of time to perform the sanitizing process, even if the second pushbutton switch 70 is not kept in a closed state by the user.

### 2. OPERATION OF THE PREFERRED EMBODIMENT

The preferred embodiment of the present invention can be utilized by the common user in a simple and effortless manner with little or no training. It is envisioned that the device 10 would be constructed in general accordance with FIG. 1 through FIG. 4. The user would procure the device 10 from conventional procurement channels such as mobile telephone 15 retailers, electronics stores, discount stores, department stores, mail order and internet supply houses and the like. Special attention would be paid to the make of model of mobile telephone 15 such that the device 10 would securely fit.

After procurement and prior to utilization, the device 10 would be prepared in the following manner: the mobile telephone 15 would be inserted into the device 10 where it is held in place via friction fit; the rechargeable battery 100 would be charged by inserting a proper power cord into the recharging port 85 for a suitable period of time; and the reservoir 75 would be filled with a suitable sanitizing solution 35 using the fill port 80 on the enclosure 45. At this point in time, the device 10 is ready for utilization.

During utilization of the device 10, the following procedure would be initiated: the user would utilize the mobile telephone 15 in an identical manner to conventional mobile telephone 15 for the purposes of phone call, text messaging, internet connectivity, picture taking, and the like; on a periodic basis, the user may activate the first pushbutton switch 65 to dispense sanitizing solution 35 on the front and rear surfaces of the mobile telephone 15 and/or the device 10 to provide for disinfecting the exterior surfaces and eradicate germs, bacteria, and viruses; the user may also activate the second pushbutton switch 70 to energize the front mounted UV-C LED's 40 and the fill port 80 on the front and rear surfaces of the mobile telephone 15 and/or the device 10 to render harmless any germs, bacteria and viruses present. The user may also activate

What is claimed is:

1. A mobile telephone case, comprising:
a case body, capable of removably retaining a mobile telephone therein, having a front side and a rear side, said front side having a front opening, said rear side having a plurality of rear openings;
a protective border located around the perimeter thereof, having a plurality of button covers;
an enclosure, further having a power supply therein;
a first sanitizing system incorporated within said case body, capable of projecting a first sanitizing element onto said front side and said back side; and,
a second sanitizing system incorporated within said case body, capable of projecting a second sanitizing element onto said front side and said back side;
wherein said first sanitizing system has a first system control means located on said enclosure in electrical communication between said first sanitizing system and said power supply; and,
wherein said second sanitizing system has a second system control means located on said enclosure in electrical communication between said second sanitizing system and said power supply.

2. The case of claim 1, wherein said first sanitizing system further comprises:
a set of first nozzles located on said front side;
a set of second nozzles located on said rear side;
a reservoir located within said enclosure and in said fluid communication with said first and second nozzles; and,
a pump in fluid communication with said reservoir and in electrical communication with said first system control means;
wherein said reservoir is capable of holding a sanitizing solution therein.

3. The case of claim 1, wherein said first sanitizing system further comprises:
a set of first UV-C LED lamps located on said front side;
a set of second UV-C LED lamps located on said rear side;
an LED drive/timer circuit in electrical communication between said first UV-C LED lamps, said second UV-C LED lamps, and said second system control means.
